(19) United States
(12) Patent Application Publication
(10) Pub. No.: US 2013/0060256 A1
(43) Pub. Date: Mar. 7, 2013

(54) FLAT-TYPE INTEGRAL PRELOADED INJECTOR WITH INTRAOCULAR LENS FIXING DEVICE

(71) Applicant: Myoung Soo Han, Cheongju-si (KR)

(72) Inventor: Myoung Soo Han, Cheongju-si (KR)

(21) Appl. No.: 13/379,563

(22) PCT Filed: Nov. 29, 2011
(86) PCT No.: PCT/KR2011/009139

§ 371 Date: Aug. 30, 2012

(30) Foreign Application Priority Data
Sep. 6, 2011 (KR) 10-2011-0090347

(51) Int. Cl. A61F 9/007

(52) U.S. Cl. 606/107

(57) ABSTRACT

An embodiment of the invention relates to a flat-type integral preloaded injector with an intraocular lens fixing device, and more particularly, to a flat-type integral preloaded injector which is provided with an intraocular lens fixing device for injecting an intraocular lens used in a cataract surgery.

### TECHNICAL FIELD

The present invention relates to a flat-type integral preloaded injector with an intraocular lens fixing device, and more particularly, to a flat-type integral preloaded injector which is provided with an intraocular lens fixing device for injecting an intraocular lens used in a cataract surgery.

### BACKGROUND ART

Typically, cataract is a disease caused by opacity of the crystalline lens of an eye and may lead to blindness in severe cases.

The cataract is classified into a congenital cataract present in utero or at birth, a senile cataract occurring in the aged, a generalized cataract caused by various diseases in the whole body, and the like. Among them, the senile cataract is most frequently observed in the aged.

Methods for treatment of cataract include pharmacotherapy and surgery. Since there is no guarantee that the pharmacotherapy can completely cure the cataract, the cataract surgery is mainly used.

Cataract surgery methods include intracapsular extraction, in which the entire crystalline lens and its enclosing capsule are removed, and extracapsular extraction, in which the posterior capsule of the crystalline lens having a thickness of about 0.01 mm, which is located adjacent to the hyaloid, is left in place, the remaining crystalline lens is removed, and an intraocular lens is inserted into place.

Here, the intraocular lens is an artificial lens used to replace the natural crystalline lens of the eye when the natural lens has cataracts or is otherwise diseased. The intraocular lens is also sometimes implanted into the eye to correct refractive errors of the eye.

The intraocular lens may be made of a variety of materials or combinations of materials such as PMMA, silicone, hydrogel, silicone hydrogel, etc.

In order to implant the intraocular lens into the eye, the sclera of the eye is incised, and a viscoelastic material is injected into the sclera to protect the anterior tissues. Then, the upper part of the lens capsule is incised to remove the clouded lens.

Subsequently, the clouded lens nucleus is completely removed from the lens capsule, and the intraocular lens is injected into the lens capsule while injecting the viscoelastic material into the lens capsule to facilitate the insertion of the intraocular lens.

At this time, the intraocular lens is injected into the lens capsule in a folded or rolled state and then restored to its original state, during which an intraocular lens injector is used.

However, according to conventional injectors, the intraocular lens should be mounted in the injector, which causes inconvenience in the use. Moreover, in order to mount the intraocular lens in the injector, the intraocular lens should be pressed with tweezers, which deforms the intraocular lens, thereby causing damage to the intraocular lens.

Furthermore, when the intraocular lens is not correctly mounted in the injector, the intraocular lens may be rotated, turned upside down, or damaged, and thus an unskilled worker cannot mount the intraocular lens in injector and thus should be well acquainted with the injection method for each injector.

### DISCLOSURE

### Technical Problem

Accordingly, the present invention has been made to solve the above-described problems, and an object of the present invention is to easily insert an intraocular lens into an eye of a patient by integrally configuring a cartridge in which the intraocular lens is mounted and a cylinder in which a plunger is operated in a sliding manner.

Moreover, another object of the present invention is to prevent the intraocular lens from entering the inside of a loading bay during transport after packaging and to firmly fix a cartridge support and a cartridge cover in such a manner that guide projections are formed on both upper and lower sides of a cartridge outlet and an intraocular lens fixing holder is attachably and detachably mounted on the guide projections.

Furthermore, still another object of the present invention is to facilitate the transport and insertion of the intraocular lens by forming a pressure holder at the end of a plunger.

### Technical Solution

To accomplish the above objects of the present invention, there is provided a flat-type integral preloaded injector with an intraocular lens fixing device for inserting an intraocular lens into an eye, the flat-type integral preloaded injector with the intraocular lens fixing device comprising: a cartridge 30 comprising a cartridge support 32 with an accommodating space 33 formed on one side of a cartridge body 31 in the longitudinal direction, a cartridge outlet 34 with a cartridge outlet passage 35 formed on one side of the cartridge support 32 to discharge an intraocular lens 20 inserted into the accommodating space 33 to the outside, and a cartridge cover 36 formed on the other side of the cartridge support 32 to rotatably operate to open and close the accommodating space 33 and including an engagement projection 37 fixedly engaged with the cartridge support 32; a cylinder 40 integrally formed with the cartridge 30 and comprising a cylinder body 41 including a moving space 42 connected to the accommodating space 33 and a plunger insertion preventing plate 43 formed vertically on the opposite side thereof; and a plunger 50 comprising a plunger body 51 having a predetermined length and slidably inserted into the cylinder 40, a pressure holder 52 formed on one side of the plunger body 51 to fixedly support the cartridge 30 in which the intraocular lens 20 inserted into the accommodating space 33 is mounted, and a plunger fixing plate 53 formed vertically on the opposite side of the plunger body 51 to be fixed by the plunger insertion preventing plate 43.

### Advantageous Effects

According to the present invention, it is possible to easily insert the intraocular lens into the eye of the patient by integrally configuring the cartridge in which the intraocular lens is mounted and the cylinder in which the plunger is operated in a sliding manner.

Moreover, it is possible to prevent the intraocular lens from entering the inside of the loading bay during transport after packaging and to firmly fix the cartridge support and the cartridge cover in such a manner that the guide projections are formed on both upper and lower sides of the cartridge outlet and the intraocular lens fixing holder is attachably and detachably mounted on the guide projections.

Furthermore, it is possible to facilitate the transport and insertion of the intraocular lens by forming the pressure holder at the end of the plunger.

### DESCRIPTION OF DRAWINGS

FIG. 1 is a perspective view showing a flat-type integral preloaded injector with an intraocular lens fixing device in accordance with the present invention.

FIG. 2 is a plan view showing the flat-type integral preloaded injector with an intraocular lens fixing device in accordance with the present invention.

FIG. 3 is a side view showing the flat-type integral preloaded injector with an intraocular lens fixing device in accordance with the present invention.

FIGS. 4 and 5 are exploded perspective views showing a cartridge and a cylinder that constitute the flat-type integral preloaded injector with an intraocular lens fixing device in accordance with the present invention.

### MODE FOR INVENTION

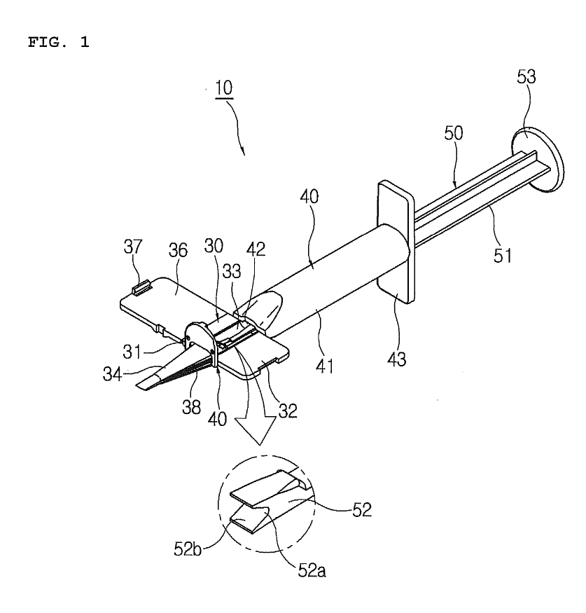

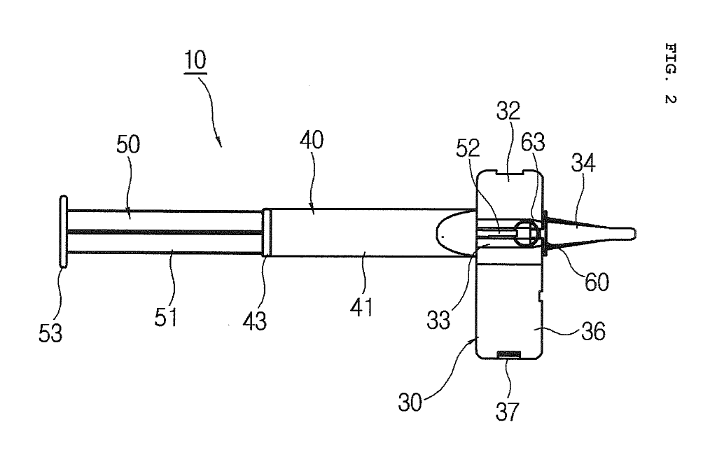

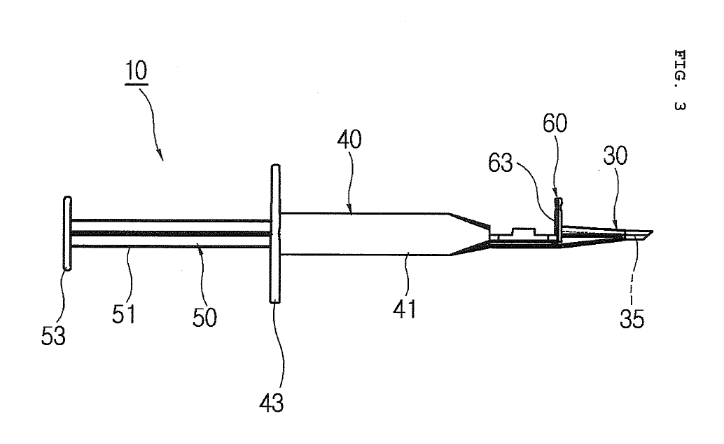

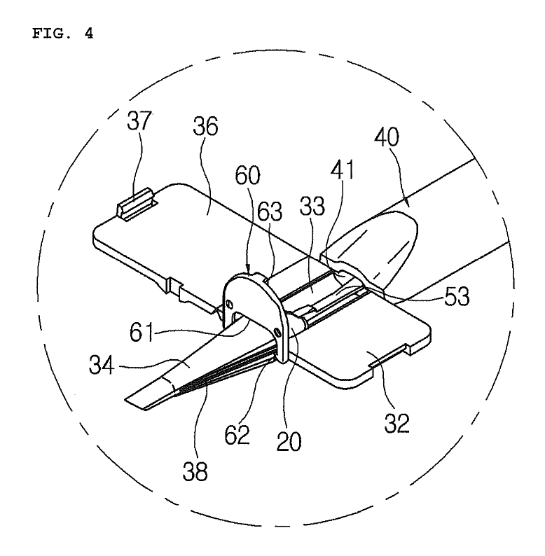

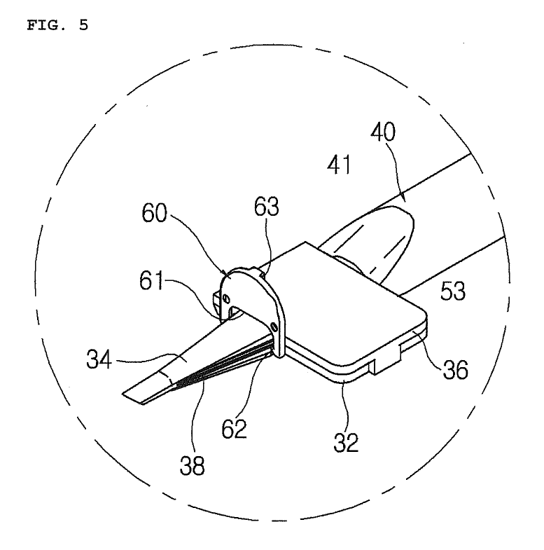

Hereinafter, preferred embodiments of the present invention will be described with reference to the accompanying drawings. FIG. 1 is a perspective view showing a flat-type integral preloaded injector with an intraocular lens fixing device in accordance with the present invention, FIG. 2 is a plan view showing the flat-type integral preloaded injector with an intraocular lens fixing device in accordance with the present invention, FIG. 3 is a side view showing the flat-type integral preloaded injector with an intraocular lens fixing device in accordance with the present invention, and FIGS. 4 and 5 are exploded perspective views showing a cartridge and a cylinder that constitute the flat-type integral preloaded injector with an intraocular lens fixing device in accordance with the present invention.

A flat-type integral preloaded injector 10 with an intraocular lens fixing device in accordance with the present invention comprises an intraocular lens 20 inserted into an eye of a patient, a cartridge 30 for accommodating and discharging the intraocular lens 20, a cylinder 40 having a predetermined length and integrally formed with the cartridge 30, and a plunger 50 inserted and mounted in the rear of the cylinder 40 and discharging the intraocular lens 20, mounted in the cartridge 30, to the outside in a sliding manner.

Here, the intraocular lens 20 is selected from those known in the art and mounted flat in a loading bay, of which detailed description will be omitted.

The cartridge 30 for accommodating and discharging the intraocular lens 20 comprises a cartridge body 31, a cartridge support 32 formed on one side of the cartridge body 31, a cartridge outlet 34 formed on the other side of the cartridge body 31, and a cartridge cover 36 formed on one side of the cartridge 30.

Moreover, the cartridge support 32 comprises an accommodating space 33 formed in the longitudinal direction of the cartridge support 32 to accommodate the intraocular lens 20 mounted in the loading bay.

Furthermore, the cartridge outlet 34 formed on the other side of the cartridge support 32 has a conical shape and comprises a cartridge outlet passage 35 formed therein and connected to the accommodating space 33 and guide projections 38 formed on both upper and lower sides at regular intervals in the longitudinal direction.

That is, the cartridge outlet 34 is configured to discharge the intraocular lens 20 accommodated in the accommodating space 33 to the outside through the cartridge outlet passage 35 and to mount an intraocular lens fixing holder 60, which will be described later, in a space formed between the guide projections 38.

The cartridge cover 36 formed on one side of the cartridge support 32 has the same size as the cartridge support 32 and comprises an engagement projection 37 formed at the end thereof to be fixedly engaged with the cartridge support 32.

That is, the cartridge cover 36 rotatably operates with respect to a portion, which is in contact with the cartridge support 32, to open and close the accommodating space 33 and is fixed to the cartridge support 32 by the engagement projection 37.

The cylinder 40 integrally formed with the cartridge 30 has a predetermined length and comprises a cylinder body 41 including a moving space 42 connected to the accommodating space 33 and a plunger insertion preventing plate 43 formed vertically on the opposite side thereof.

The plunger 50 inserted into the cylinder 40 and operated in a sliding manner comprises a plunger body 51 having a predetermined length, a pressure holder 52 formed on one side of the plunger body 51 and fixedly supporting the cartridge 30 in which the intraocular lens 20 is mounted, and a plunger fixing plate 53 formed vertically on the opposite side of the plunger body 51.

That is, when an external force is applied to the plunger fixing plate 53, the plunger 50 is operated in a sliding manner such that the pressure holder 52 guides the intraocular lens 20 placed in the accommodating space 33 to the cartridge outlet 34 to be discharged to the outside.

Here, the pressure holder 52 that fixedly presses the intraocular lens 20 has a structure in which an accommodating portion 52a formed in the center thereof is depressed inward in the longitudinal direction and an upper surface 52b and a lower surface of the accommodating portion 52a are inclined to smoothly accommodate the intraocular lens 20.

In more detail, the end of the pressure holder 52 has a shape of “Σ” such that the accommodating portion 52a fixes the intraocular lens 20 located in the accommodating space 33.

The intraocular lens fixing holder 60, which is mounted in the space formed between the guide projections 38 and fixes the cartridge support 32 and the cartridge cover 36, comprises an accommodating groove 61 formed in the lower center thereof, insertion projections 62 symmetrically formed on both sides of the accommodating groove 61 to be inserted into the space formed between the guide projections 38, and a vertical fixing member 63 integrally formed in the inside thereof to fix the cartridge support 32 and the cartridge cover 36.

That is, the intraocular lens fixing holder 60 serves to prevent the intraocular lens 20 from entering the inside of the loading bay during transport after packaging in such a manner that, while the accommodating groove 61 accommodates the cartridge outlet 34, the insertion projections 62 are inserted and fixed into the space formed between the guide projections 38, and the vertical fixing member 63 fixes the cartridge support 32 and the cartridge cover 36.

The flat-type integral preloaded injector with the intraocular lens fixing device in accordance with the present invention having the above-described configuration will be described with reference to an example below.

First, the intraocular lens fixing holder 60, in which the accommodating groove 61 is formed in the lower center thereof, the insertion projections 62 are symmetrically formed on both sides of the accommodating groove 61, and the vertical fixing member 63 is integrally formed in the inside thereof, is provided.

Then, the cartridge 30, in which the cartridge support 32 with the accommodating space 33 is formed on one side of the cartridge body 31 in the longitudinal direction, the cartridge outlet 34 with the cartridge outlet passage 35 formed to discharge the intraocular lens 20 inserted into the accommodating space 33 to the outside is formed on one side of the cartridge support 32, the cartridge cover 36 is formed on the other side of the cartridge support 32 to rotatably operate to open and close the accommodating space 33 and including the engagement projection 37 fixedly engaged with the cartridge support 32, and the guide projections 38 are formed on both upper and lower sides of the cartridge outlet 34, is integrally formed with the cylinder 40, in which the moving space 42 of the cylinder body 41 is connected to the accommodating space 33 and the plunger insertion preventing plate 43 is formed vertically on the opposite side thereof.

Next, the plunger 50, in which the plunger body 51 having a predetermined length is formed, the pressure holder 52 is formed on one side of the plunger body 51 to fixedly support the cartridge 30 in which the intraocular lens 20 inserted into the accommodating space 33 is mounted, and the plunger fixing plate 53 is formed vertically on the opposite side of the plunger body 51 to be fixed by the plunger insertion preventing plate 43.

Subsequently, the plunger 50 is inserted and mounted in the cylinder 40, and the intraocular lens fixing holder 60 is mounted in the space formed between the guide projections 38 formed on both upper and lower sides of the cartridge outlet 34, thereby completing the assembly of the flat-type integral preloaded injector with the intraocular lens fixing device.

Here, the assembly sequence of the flat-type integral preloaded injector with the intraocular lens fixing device may be configured in a manner different from that described above.

The insertion operation of the intraocular lens into the eye of the patient using the flat-type integral preloaded injector with the intraocular lens fixing device is as follows.

First, the intraocular lens 20 mounted flat on the top of the loading bay is mounted in the accommodating space 33 that constitutes the cartridge 30. Here, one side of the intraocular lens 20 is fixed to the accommodating portion 52a of the pressure holder 52 that constitutes the plunger 50.

Then, the cartridge cover 36 that constitutes the cartridge 30 is rotatably operated to be fixedly engaged with the cartridge support 32 by the engagement projection 37.

Subsequently, the intraocular lens 20 accommodated in the accommodating space 33 of the cartridge 30 is inserted into the eye of the patient by pressing the plunger 50 to the cylinder 40, thereby completing the insertion of the intraocular lens 20.

That is, when the plunger 50 is pressed, the pressure holder 52 of the plunger body 51 moves forward such that the cartridge 30 mounted with the intraocular lens 20 is delivered to the cartridge outlet 34, thereby discharging the intraocular lens 20 to the outside.

While the preferred embodiments of the present invention have been illustrated and described, it will be understood by those of ordinary skill in the art that changes and other modifications can be made without departing from the invention in its broader aspects. Various features of the present invention are set forth in the following claims.

1. A flat-type integral preloaded injector with an intraocular lens fixing device for inserting an intraocular lens into an eye, the flat-type integral preloaded injector comprising:
a cartridge comprising a cartridge support with an accommodating space formed on one side of a cartridge body in the longitudinal direction, a cartridge outlet with a cartridge outlet passage formed on one side of the cartridge support to discharge an intraocular lens inserted into the accommodating space to the outside, and a cartridge cover formed on the other side of the cartridge support to rotatably operate to open and close the accommodating space and including an engagement projection fixedly engaged with the cartridge support;
a cylinder integrally formed with the cartridge and comprising a cylinder body including a moving space connected to the accommodating space and a plunger insertion preventing plate formed vertically on the opposite side thereof; and
a plunger comprising a plunger body having a predetermined length and slidably inserted into the cylinder, a pressure holder formed on one side of the plunger body to fixedly support the cartridge in which the intraocular lens inserted into the accommodating space is mounted, and a plunger fixing plate formed vertically on the opposite side of the plunger body to be fixed by the plunger insertion preventing plate.

2. The flat-type integral preloaded injector with the intraocular lens fixing device of claim 1, wherein the cartridge outlet comprises guide projections formed on both upper and lower sides thereof at regular intervals in the longitudinal direction and an intraocular lens fixing holder attachably and detachably mounted between the guide projections to fix the cartridge support and the cartridge cover.

3. The flat-type integral preloaded injector with the intraocular lens fixing device of claim 2, wherein the intraocular lens fixing holder comprises an accommodating groove formed in the lower center thereof, insertion projections symmetrically formed on both sides of the accommodating groove to be inserted into a space formed between the guide projections, and a vertical fixing member integrally formed in the inside thereof to fix the cartridge support and the cartridge cover.

4. The flat-type integral preloaded injector with the intraocular lens fixing device of claim 1, wherein the pressure holder comprises an accommodating portion formed in the center thereof and depressed inward in the longitudinal direction to facilitate the insertion and fixing of the intraocular lens, the accommodating portion comprising an upper surface and a lower surface formed to be inclined.
